(19) United States
(12) Patent Application Publication
(10) Pub. No.: US 2018/0120282 A1
(43) Pub. Date: May 3, 2018

(54) SYSTEM AND METHOD FOR VEHICLE GAS FUEL MANAGEMENT

(71) Applicant: GM GLOBAL TECHNOLOGY OPERATIONS LLC, DETROIT, MI (US)

(72) Inventor: Remington C. Booher, Oshawa (CA)

(21) Appl. No.: 15/338,828

(22) Filed: Oct. 31, 2016

(51) Int. Cl. G01N 33/22; F02M 37/00; G01N 25/00

(52) U.S. Cl. CPC G01N 33/225; B60K 2015/03144; G01N 25/00; F02M 37/0088

(57) ABSTRACT

A vehicle gas fuel management system includes an ambient temperature sensor that detects a first ambient temperature at a first time and a second ambient temperature at a second time, a pressure sensor that detects a pressure for a volume of gas at the first time, and a controller. The controller is programmed to determine a first enthalpy and density for the volume of gas based upon the detected first ambient temperature and detected pressure, and estimate a second enthalpy for the volume of gas based upon the second ambient temperature and the first energy state.

### FIELD

The present disclosure relates to a system and method for vehicle gas fuel management.

### INTRODUCTION

This introduction generally presents the context of the disclosure. Work of the presently named inventors, to the extent it is described in this introduction, as well as aspects of the description that may not otherwise qualify as prior art at the time of filing, are neither expressly nor impliedly admitted as prior art against this disclosure.

Vehicles that are powered by fuel that is stored in a gas state, as opposed to a liquid state, such as, for example, compressed natural gas (CNG), may become more common as viable alternative energy sources are explored. In order to store CNG in a vehicle fuel tank, the gas fuel is pressurized in a tank in the vehicle. The gas fuel is delivered to a vehicle engine by a delivery system which, generally, depressurizes the fuel and injects the fuel into the engine.

The management of fuel in a compressed gas fuel system for a vehicle may pose several challenges. Unlike a liquid, which is generally incompressible, a gas fuel will behave differently according to the conditions experienced by that gas fuel. For example, a gas will behave differently according to the pressure, temperature and the volume of the container which contains the gas. In general, the gas in a gas fuel system will act in accordance with the equation:

Pv=nRT (1)

Where P is the pressure, v is the volume of the gas, n is the number of moles of gas (i.e. the quantity of gas), R is a gas constant (preferably the specific gas constant), and T is the temperature. Thus, for example, when the volume remains constant, if the temperature increases, the pressure of the gas will also increase. Further, in order to know the quantity of the gas (i.e. the number of moles), the volume, the temperature and the pressure must be known.

Vehicles are exposed to widely varying temperatures and those temperatures are generally changing (i.e. rarely held constant). Further, as the vehicle consumes the gas fuel in the engine and/or the gas fuel is replenished in a refilling operation, the quantity of the fuel changes. Of course, as the temperatures vary and the quantity varies, the pressure of the gas will also vary. This makes it difficult to determine the conditions of the gas fuel in a vehicle gas fuel system during non-steady state, varying conditions. It is desirable to know the conditions of the gas fuel in such a system to avoid over-pressure conditions, detect leaks, determine vehicle range, and the like.

Conventionally, vehicle gas fuel management systems are only able to reliably determine the conditions of a gas fuel after the vehicle has experienced unchanging temperatures and not experiencing mass flows between various containers within the system for a long enough time for conditions within the system to stabilize (a “soak” time). After that predetermined period of time has elapsed, a pressure and temperature measurement may be taken to reliably determine the current conditions and quantity of fuel. In between these times of stability, the conditions can widely fluctuate and any measurement of temperature and pressure during these varying conditions that might lead to any estimate of the conditions are largely unreliable. In those situations, conventional vehicle gas fuel management system are required to provide very wide boundary conditions on their estimates of those conditions.

Additionally, pressure and temperature sensors in these vehicle gas fuel systems are not typically positioned within each identifiable volume within that system. To do so would be quite expensive.

Further, even if no expense was spared and sensors were placed in every possible identifiable volume within the gas fuel system, the sensed temperatures would not be reliable. For example, while a temperature sensor may be positioned within a gas fuel tank the actual temperature that is sensed may be more indicative of the temperature of the wall of the pressure vessel containing the gas fuel than the gas fuel itself. Additionally, temperatures within any given volume of a gas fuel may vary widely. Thus, resorting to an attempt to provide a more reliable estimate of the conditions of gas fuel in a vehicle by providing a plurality of sensors, is not only expensive, but also not reliable.

### SUMMARY

In an exemplary aspect, a vehicle gas fuel management system includes an ambient temperature sensor that detects a first ambient temperature at a first time and a second ambient temperature at a second time, a pressure sensor that detects a pressure for a volume of gas at the first time, and a controller that is programmed to determine a first enthalpy and density for the volume of gas based upon the detected first ambient temperature and detected pressure, and estimate a second enthalpy for the volume of gas based upon the second ambient temperature and the first energy state.

In another exemplary aspect, the controller is further programmed to estimate the second enthalpy by estimating an amount of heat transfer between the volume of gas and an ambient environment.

In another exemplary aspect, the controller is further programmed to estimate the amount of heat transfer based upon a speed of a vehicle that includes the vehicle gas fuel management system.

In another exemplary aspect, the controller is further programmed to estimate a transfer of mass between the volume of gas and another volume of gas within the vehicle fuel system.

In another exemplary aspect, the controller is further programmed to receive a mass flow signal representing a change of mass for the volume of gas.

In another exemplary aspect, the controller is programmed to estimate the second enthalpy based upon the mass flow signal.

In another exemplary aspect, the mass flow signal represents a mass flow from the volume of gas consumed by an internal combustion engine in the vehicle.

In another exemplary aspect, the controller is further programmed to estimate the temperature for the volume of gas at the second time based upon the estimated second enthalpy.

In another exemplary aspect, the controller is further programmed to estimate a pressure for the volume of gas at the second time based upon the estimated second enthalpy.

In another exemplary aspect, the controller is further programmed to estimate a density for the volume of gas at the second time based upon the estimated second enthalpy.

In this manner, conditions of gaseous fuel within a vehicle fuel system may be accurately estimated. In particular, determining the enthalpy and mass of various volumes of gaseous fuel within the vehicle fuel system and adjusting estimates for the enthalpy and mass based upon detected ambient temperature enables a real-time accurate condition estimation of that gaseous fuel during varying conditions and in-between soak times. Additionally, with improved and more reliable understanding of the conditions within the fuel system, error bounds may be significantly reduced.

Further areas of applicability of the present disclosure will become apparent from the detailed description provided below. It should be understood that the detailed description and specific examples are intended for purposes of illustration only and are not intended to limit the scope of the disclosure.

The above features and advantages, and other features and advantages, of the present invention are readily apparent from the detailed description, including the claims, and exemplary embodiments when taken in connection with the accompanying drawings.

### BRIEF DESCRIPTION OF THE DRAWINGS

The present disclosure will become more fully understood from the detailed description and the accompanying drawings, wherein:

FIG. 1 shows a vehicle having a fuel system in accordance with an exemplary embodiment of the present disclosure;

FIG. 2 is a schematic diagram of a fuel system in accordance with an exemplary embodiment of the present disclosure;

FIG. 3 is a functional block diagram of an exemplary controller in accordance with an exemplary embodiment of the present disclosure; and

FIG. 4 is a flowchart illustrating an exemplary method for estimating a condition of a vehicle fuel system.

### DETAILED DESCRIPTION

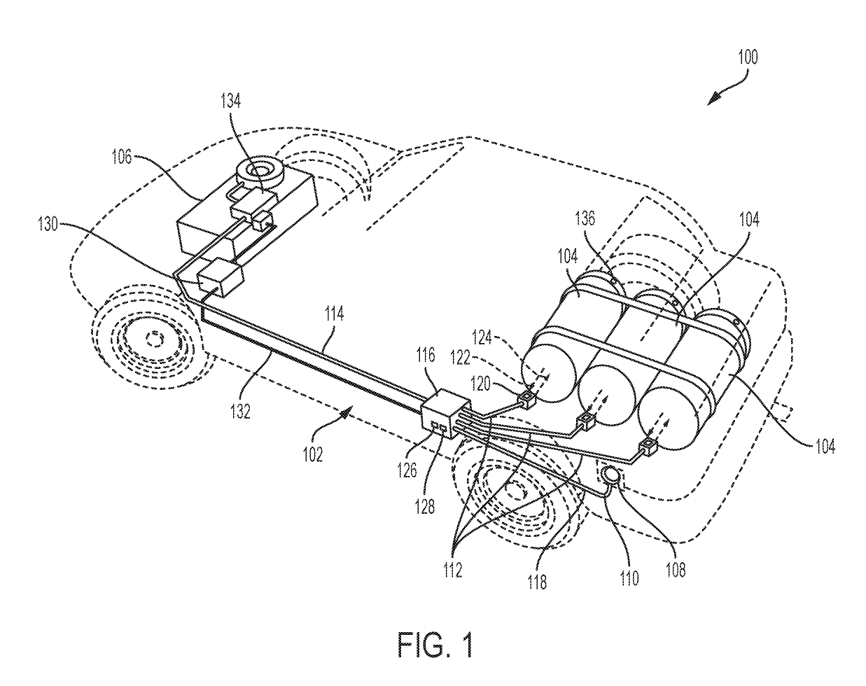

Referring to FIG. 1, a vehicle 100 having a fuel system 102 in accordance with an exemplary embodiment of the present disclosure is illustrated. The fuel system 102 includes a plurality of tanks 104 that may be filled with a gas fuel, such as, for example, compressed natural gas (CNG). The vehicle includes an engine 106 for combusting the gas fuel and providing motive power to the vehicle drivetrain (not shown). Although three tanks 104 are illustrated in FIG. 1, the vehicle 100 may include any number of a plurality of tanks or a single tank 104.

The fuel system 102 includes an inlet 108 which may be used to refill the fuel system 102 with gas fuel. The fuel system 102 includes a plurality of fuel lines 110, 112, and 114. The fuel lines 110, 112, and 114 may be high pressure gas fuel lines. Fuel line 110 is a refueling line 110 in communication with the inlet 108, the fuel lines 112 are each connected to a corresponding tank 104 (also known as “tank lines”), and fuel line 114 communicates with a fuel injector (not illustrated) for injecting the gas fuel into the engine 106. Fuel lines 110, 112, and 114 may communicate with pressure regulator 116. The refueling line 110 may also include a one-way valve 118 to prevent gas fuel from flowing from the gas fuel system 102 out of the inlet 108.

Further, valves 120, 122, and 124 may be present at each of the tanks 104 with which the flow of gas into and/or out of each tank may be independently controlled. This may include a manually operable valve 120, an electronically controllable shut-off valve 122, and a one-way valve 124. The one-way valve 124 may permit gas to flow into the tank 104 when the pressure is higher in the fuel line 112 than in the tank 104. The shut-off valve 122 selectively controls when gas fuel may leave the tank 104 and enter into the corresponding fuel line 112. Although, the one-way valve 124 and shut-off valve 122 are shown separately, they may preferably be configured in a single valve providing both functions.

The pressure regulator 116 may include a pressure sensor 126 and a temperature sensor 128. A controller 130 communicates with the pressure regulator 116 via communication line 132. The control 132 may also communicate and control shut-off valves 122. The controller 130 also may read signals from pressure sensor 126 and temperature sensor 128. The controller 130 may also communicate with an engine control module 134. Although, FIG. 1 shows controller 130 and engine control module 134 separately, controlling the fuel system 102 in accordance with the present disclosure may be directly affected by the engine control module 134 without a separate controller 130 being present. Further controllers (not shown) may also be present in the vehicle and a controller area network (not shown) may be provided to enable communication between the controllers.

The fuel system 102 may also include a high-pressure safeguard which ensures that any overpressure that may develop in a tank 104 may be relieved. The high-pressure safeguard may include a pressure-relief disc 136.

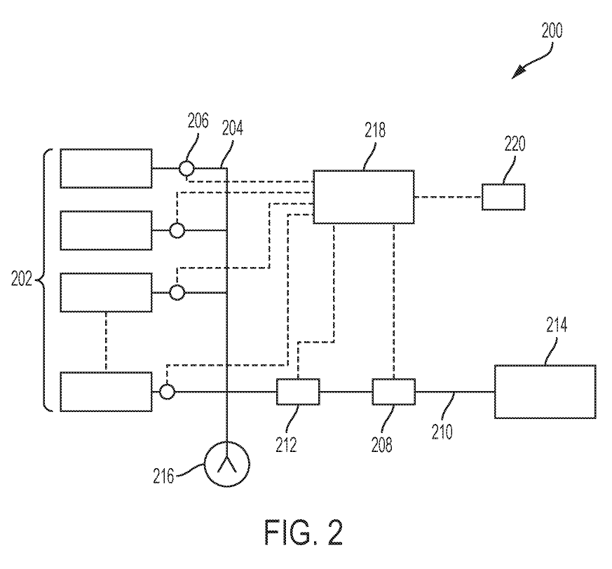

FIG. 2 schematically illustrates an exemplary fuel system 200 in accordance with an exemplary embodiment of the present disclosure. The fuel system 200 includes a plurality of tanks 202. As explained above, any number of tanks may be present in the fuel system 200 without limitation and form an exemplary embodiment of the present invention without limitation. Each of the plurality of tanks 202 communicates with tank line 204 via a shut-off valve 206. The tank line 204 communicates with a pressure regulator 208 to a fuel supply line 210. The pressure of the gas fuel in the fuel supply line 210 is regulated by a pressure regulator 208. The pressure regulator 208 may include a pressure sensor 212, or alternatively, the pressure sensor 212 may be independent of the pressure regulator 208. The pressure sensor 212 measures the pressure in the tank line 204. The fuel supply line 210 supplies fuel from the regulator 208 to an engine 214. An alternative exemplary embodiment for a fuel system (not shown) may also include a rail that extends to injectors to inject the gas fuel into the engine 214. This rail may also include a temperature sensor and a pressure sensor.

The fuel system 200 further includes an intake valve 216 which is preferably configured as a one-way valve that only permits flow of gas fuel into the fuel system 200 and not out. The fuel system also includes a controller 218 and an ambient temperature sensor 220. The ambient temperature sensor 220 senses the temperature of the environment in which the fuel system 200 may presently reside. The ambient temperature sensor 220 may be positioned anywhere within the system 200 or vehicle without limitation. The controller 218 communicates with the ambient temperature sensor 220 and the pressure sensor 212 to receive measurement signals. The controller 218 also communicates with and may control the pressure regulator 208 and the shut off valves 206 as well as other components in the vehicle without limitation. Preferably, the controller 218 controls each shut off valve 206 independently. Further, as explained above, the controller 218 may communicate with other processors and/or controllers (not shown) via a controller area network (not shown) without limitation.

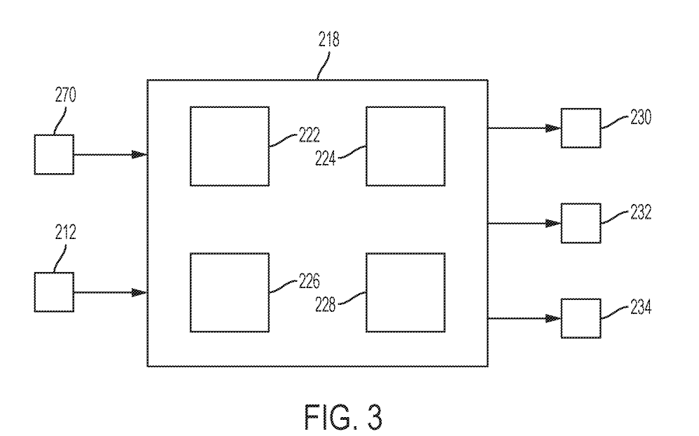

FIG. 3 is a functional block diagram of an exemplary controller 218 in accordance with an exemplary embodiment of the present invention. As explained above, the controller 218 communicates with the ambient temperature sensor 220 and the pressure sensor 212. The controller 218 may also communicate with a vehicle speed sensor (not shown) to receive a signal indicative of the speed of the vehicle. The controller 218 is programmed to include an enthalpy determination module 222, a heat transfer estimation module 224, a mass flow estimation module 226, and an enthalpy estimation module 228. The controller 218 outputs an estimated temperature value 230, and an estimated pressure value 232. Optionally, the controller 218 may further output other signals indicating an estimated condition of the gas fuel in the fuel system 200, such as, for example, an estimated enthalpy value 234 or an estimated density, without limitation and form an exemplary embodiment of the present invention.

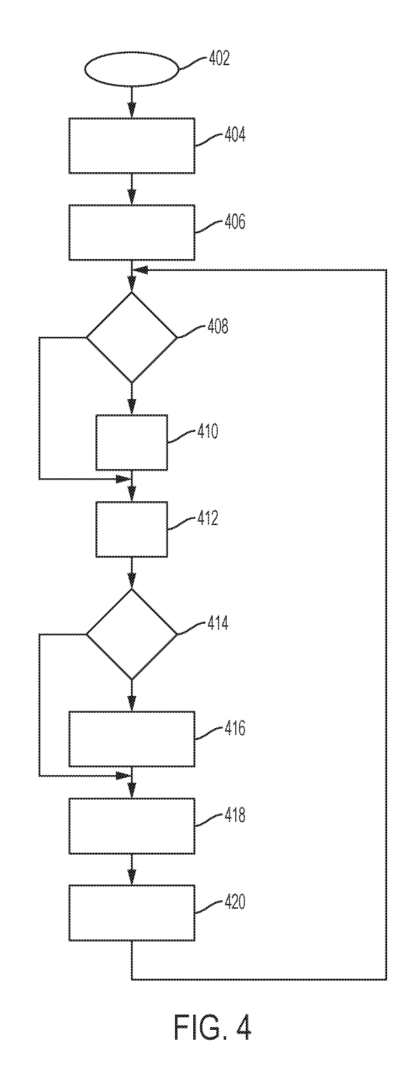

FIG. 4 is a flowchart illustrating an exemplary method 400 for estimating a condition of the vehicle fuel system 200 in accordance with the present disclosure. The method starts at step 402 and continues to step 404 where the controller 218 receives a temperature signal from the ambient temperature sensor 220 and a pressure signal from the pressure sensor 212. Preferably, these values are received after a predetermined period of time has elapsed where these values have stabilized. For example, the method may optionally further determine whether a predetermined time has elapsed under steady-state conditions (also called a “soak time”). In this manner, the method obtains reliable initial conditions after the fuel system has stabilized and experiencing steady-state conditions. The method then continues to step 406.

In step 406, the enthalpy determination module 222 of the controller 218 determines initial values (conditions) for the enthalpy and density for each separate and independent volume within the fuel system 200. Optionally, the controller 218 may also determine the mass of the gas within each volume. The enthalpy determination module 222 may determine the enthalpy and density using look up tables and/or by direct calculation using equation (1), listed above, and:

H=U+PV (2)

Where H is the enthalpy, U is the internal energy, P is pressure, and V is the volume. The internal energy U is determined from the relation:

U=cNT (3)

Where c is the heat capacity of the gas, N is the number of moles, and T is the temperature.

Optionally and preferably, the controller 218 may also determine the density of the gas within each volume. Density may be calculated by:

D=m/V

Where D is the density, m is the mass of the gas, and V is the volume of the container.

These values may be stored in a data storage (not shown) and/or provided on a controller area network (CAN) to enable several potentially useful functions to be performed, such as, for example, determining whether an over-pressure condition exists, determining whether a leak is occurring, determining the remaining fuel range, predicting a possible over-pressure condition if the vehicle were to experience an increase in ambient temperature and/or the like without limitation.

The method continues to step 408 where the mass flow module 226 of the controller 218 determines whether mass transfer between the separate and independent volumes within the fuel system 200, into, and/or out of the fuel system is occurring. Mass flow module 226 may make these determinations in several ways. For example, the mass flow module 226 may communicate with an engine control unit 134 (ECU) which may communicate the mass flow of gas being consumed by the engine 214 and, thus, leaving the fuel system 200 or leaving the fuel supply line 210. If, in step 408, the method determines that mass flow is occurring, the method continues to step 410.

The mass flow module 226 may also determine a mass flow of gas fuel between separate and independent volumes within the fuel system 200. For example, a mass of gas fuel may be flowing from the tank line 204 into one or more of the tanks 202 in the instance where the ambient temperature may have risen, such as when, the vehicle is driven from outside in cold temperatures into a warm garage and one or more of the shut off valves 206 is open or a by-pass valve 206 operates to enable a higher pressure flow from the tank line 204 into a tank 202. In this instance, the temperature of the gas fuel in the tank line 204 may increase more rapidly than the gas fuel in a tank 204. In this situation, the increase in temperature more quickly increasing in the tank line 204 than a tank 202 will cause a corresponding more rapid increase in pressure in the tank line 204 than in the tank 202, which will result in a mass flow of gas fuel from the tank line 204 to a tank 202.

In step 410, the controller 218 determines the direction and the amount of mass flowing between the various volumes within the fuel system 200 and continues to step 412. Alternatively, if in step 408, the method determines that no mass transfer is occurring between the various volumes, then the method continues to step 412.

Exemplary mass flows between volumes of the fuel system 200 may include, for example, a flow of mass from the tank line 204 to a tank 202, from the tank line 204 to the engine 214, from a tank 202 to the tank line 204, and from anywhere within the fuel system 200 and external to the fuel system 200 (for example a leak), without limitation.

In step 412, the controller 218 receives an updated temperature signal from the ambient temperature sensor 220 and continues to step 414. Optionally, the controller 218 may also receive a vehicle speed signal indicating a speed of the vehicle. In step 414, the heat transfer module 224 of the controller 218 determines whether heat transfer is occurring between the various volumes within the fuel system 200 and the ambient environment. The “various volumes” may include, for example, a tank 202, the fuel line 204, the fuel supply line 210 and the like without limitation. In particular, the heat transfer module 224 determines whether there is a temperature differential between each of the various volumes within the fuel system 200. If there is a difference in temperature, then there will be heat transfer. If, in step 414, the heat transfer module 224 determines that heat transfer is occurring, then the method continues to step 416.

In step 416, the method determines the direction and amount of heat being transferred between each of the various volumes within the fuel system 200 and the ambient environment. The heat transfer module 224 may make this determination using a look-up table and/or by reference to heat transfer equations. The direction of the heat transfer is from a higher temperature to a lower temperature. The heat transfer equations from which the amount of heat being transferred may be estimated are well known to those of ordinary skill in the art and may be constructed having knowledge of the specific structure and accounting for each relevant mode of heat transfer (such as, for example, conduction, convection, radiation, and the like without limitation) for each relevant volume within the fuel system 200. In general, the amount of heat transfer depends upon the magnitude of the temperature difference, the shape and dimensions of the container, the speed of the vehicle (to determine that portion of heat transfer via convection) and the materials (e.g., the thermal conductivity of the walls of tank line 204 and/or the tanks 202). The method then continues to step 418.

Alternatively, if in step 414, the heat transfer module 224 determines that heat is not transferring between the ambient environment and each of the various relevant volumes within the fuel system 200, then the method continues to step 418.

In step 418, the enthalpy estimator module 228 may estimate the enthalpy for the gas fuel contained within each of the various relevant volumes within the fuel system 200. The enthalpy estimator module 228 may estimate the enthalpy using a look-up table and/or reference to equations representing the conservation of energy and mass and in reliance upon the amount of heat transfer determined above. These equations are well known and understood by those of ordinary skill in the art. The method may then continue to step 420. In step 420, the controller 418 may estimate the temperature, pressure, mass, density, and internal energy for the gas fuel within each of the various relevant volumes within the fuel system 200. The method may then return to step 408.

The steps 408 through 420 may be iteratively repeated to update and continuously estimate the changing conditions within the fuel system 200 and provide a reliable estimate of those conditions. At any time, when a predetermined amount of time elapses such that conditions within the system are stable for a long enough soak time, the entire method 400 may be re-started at step 402. For example, when the vehicle is turned on, an engine off timer may be consulted to determine whether the vehicle has been turned off and not operated for a sufficiently long, predetermined amount of time to restart the method at step 402.

Further, although the flow chart of FIG. 4 illustrates a method where steps 408, 410, and 412 are executed sequentially before steps 414, 416, and 418, these groups of steps are independent and may also be executed simultaneously or in parallel and form another exemplary embodiment of the present invention.

In this manner, using an initial measurement of temperature and pressure after a predetermined soak time, the controller 218 is able to estimate the conditions present within each volume within the fuel system using the ambient temperature signals from the ambient temperature sensor 220 and mass flow out of the system that is being consumed by the engine 214. The fuel system 200 is thereby capable of being successfully managed over an extended period of time (for example several days) without requiring a long soak time to provide reliable estimates of the conditions of the gas fuel. The controller 218 is able to make these estimations by modelling the method as a system boundary situation. The system may be limited to considering only the tank 202 and tank line 204 volumes, the mass being moved between these volumes and the engine 214, the energy and mass transferred between the volumes and the ambient environment, the conditions within the fuel system may be accurately and reliably estimated.

The inventive system and method is capable of providing accurate and reliable condition reports without requiring an extensive and expensive use of a plurality of temperature and/or pressure sensors positioned throughout the various volumes within the fuel system. Further, as explained above, even with the use of sensors, a temperature sensor will not give accurate and reliable temperature readings that are relevant across an entire mass of gas within any given volume. In contrast, average temperature estimations work very well with gas system equations when modelling the overall energy and mass balance within any given volume of gas. Thus, the exemplary embodiments of the present disclosure may work better and be more reliable than if actual sensors were used.

Additionally, in a fuel system having a plurality of sensors positioned throughout a fuel system, the exemplary systems and methods of the present disclosure may provide an accurate and reliable way to rationalize the sensor signals being generated by those sensors. For example, sensors may experience drift in their output which could be corrected when used in combination with the exemplary systems and methods of the present disclosure.

Conventional vehicle fuel system leak detection systems determine initial conditions after a long soak time and then wait until experiencing a second soak time at which time another set of condition measurements are taken. By analyzing the change in conditions at each soak time, any difference between the expected conditions and actual conditions leads to a determination that not all of the mass of the gas has been accounted for and a leak may be possible. In stark contrast, the present invention does not need to wait until experiencing a second soak time. Rather, the inventive system and method is able to provide reliable and accurate estimates of the conditions within the gas fuel system by iteratively estimating heat transfer and mass transfer continuously in varying conditions.

This description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims.

What is claimed is:

1. A vehicle gas fuel management system, comprising:
an ambient temperature sensor that detects a first ambient temperature at a first time and a second ambient temperature at a second time;
a pressure sensor that detects a pressure for a volume of gas at the first time; and
a controller that is programmed to:
determine a first enthalpy and density for the volume of gas based upon the detected first ambient temperature and detected pressure; and
estimate a second enthalpy for the volume of gas based upon the second ambient temperature and the first energy state.

2. The system of claim 1, wherein the controller is further programmed to estimate the second enthalpy by estimating an amount of heat transfer between the volume of gas and an ambient environment.

3. The system of claim 2, wherein the controller is further programmed to estimate the amount of heat transfer based upon a speed of a vehicle that includes the vehicle gas fuel management system.

4. The system of claim 1, wherein the controller is further programmed to estimate a transfer of mass between the volume of gas and another volume of gas within the vehicle fuel system.

5. The system of claim 1, wherein the controller is further programmed to receive a mass flow signal representing a change of mass for the volume of gas.

6. The system of claim 5, wherein the controller is programmed to estimate the second enthalpy based upon the mass flow signal.

7. The system of claim 5, wherein the mass flow signal represents a mass flow from the volume of gas consumed by an internal combustion engine in the vehicle.

8. The system of claim 1, wherein the controller is further programmed to estimate the temperature for the volume of gas at the second time based upon the estimated second enthalpy.

9. The system of claim 1, wherein the controller is further programmed to estimate a pressure for the volume of gas at the second time based upon the estimated second enthalpy.

10. The system of claim 1, wherein the controller is further programmed to estimate a density for the volume of gas at the second time based upon the estimated second enthalpy.

11. A method for estimating a condition of a fuel system for a vehicle comprising:
detecting a first ambient temperature at a first time by an ambient temperature sensor in the vehicle;
detecting a pressure for a volume of gas at the first time by a pressure sensor in the vehicle;
determining a first enthalpy and density for the volume of gas based upon the detected first ambient temperature and detected pressure with a controller in the vehicle;
detecting a second ambient temperature at a second time by the ambient temperature sensor; and
estimating a second enthalpy for the volume of gas based upon the second ambient temperature and the first energy state with the controller in the vehicle.

12. The method of claim 11, wherein estimating the second enthalpy comprises estimating an amount of heat transfer between the volume of gas and an ambient environment.

13. The method of claim 12, wherein estimating the amount of heat transfer is based upon a speed of a vehicle that includes the vehicle gas fuel management system.

14. The method of claim 11, further comprising estimating a transfer of mass between the volume of gas and another volume of gas within the fuel system.

15. The method of claim 11, further comprising receiving a mass flow value representing a change of mass for the volume of gas.

16. The method of claim 15, wherein estimating the second enthalpy is based upon the mass flow value.

17. The method of claim 15, wherein the mass flow value represents a mass flow from the volume of gas consumed by an internal combustion engine in the vehicle.

18. The method of claim 11, further comprising estimating temperature for the volume of gas at the second time based upon the estimated second enthalpy.

19. The method of claim 11, further comprising estimating a pressure for the volume of gas at the second time based upon the estimated second enthalpy.

20. The method of claim 11, further comprising estimating a density for the volume of gas at the second time based upon the estimated second enthalpy.
